(19) United States
(12) Patent Application Publication
(10) Pub. No.: US 2020/0182885 A1
(43) Pub. Date: Jun. 11, 2020

(54) A METHOD FOR DIAGNOSING OR MONITORING KIDNEY FUNCTION OR DIAGNOSING KIDNEY DYSFUNTION

(71) Applicant: SphingoTec GmbH, Hennigsdorf (DE)

(72) Inventor: Andreas BERGMANN, Berlin (DE)

(73) Assignee: SphingoTec GmbH, Hennigsdorf (DE)

(21) Appl. No.: 16/617,646

(22) PCT Filed: May 29, 2018
(86) PCT No.: PCT/EP2018/064049

§ 371 Date: Nov. 27, 2019

(30) Foreign Application Priority Data
May 30, 2017 (EP) 17173482.5

(51) Int. Cl. G01N 33/68

(52) U.S. Cl. CPC G01N 33/6893; G01N 2800/52; G01N 2800/347

(57) ABSTRACT

Subject matter of the present invention is a method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting incidence of (chronic) kidney disease comprising determining the level of Pro-Tachykinin A (PTA).

Subject matter of the present invention is a method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting incidence of (chronic) kidney disease comprising
determining the level of Pro-Tachykinin A (PTA) or fragments thereof of at least 5 amino acids in a bodily fluid obtained from said subject; and
(a) correlating said level of Pro-Tachykinin A or fragments thereof with kidney function in a subject, or
(b) correlating said level of Pro-Tachykinin A or fragments thereof with kidney dysfunction wherein an elevated level above a certain threshold is predictive or diagnostic for kidney dysfunction in said subject, or
(c) correlating said level of Pro-Tachykinin A or fragments thereof with said risk of an adverse event in a diseased subject, wherein an elevated level above a certain threshold is predictive for an enhanced risk of said adverse events, or
(d) correlating said level of Pro-Tachykinin A or fragments thereof with success of a therapy or intervention in a diseased subject, wherein a level below a certain threshold is predictive for a success of therapy or intervention, or
(e) predicting incidence of (chronic) kidney disease.

Subject matter of the present invention is the use of Pro-Tachykinin A (PTA) or fragments thereof as marker for kidney function and dysfunction and its clinical utility in healthy and diseased subjects. Subject matter of the present invention is a method for diagnosing or monitoring kidney function in subject or diagnosing kidney dysfunction in a subject or predicting the risk of death or adverse events or predicting or monitoring the success of a therapy or intervention or predicting incidence of (chronic) kidney disease in a diseased subject.

Kidney function has an impact on hemodynamic, vascular, inflammatory and metabolic disease due to its role in circulation and consequentially a decreased kidney function is associated with an increased risk of cardiovascular events, hospitalization and death. Thus, screening and early detection of decreased kidney function is important and therefore screening of certain risk groups, such as subjects with family predisposition as well as of patients with diabetes, hypertension, cardiovascular disease, autoimmune diseases and persons with structural disease of the renal tract is recommended.

Substance P (SP) is a neuropeptide: an undecapeptide that functions as a neurotransmitter and as a neuromodulator. It belongs to the tachykinin neuropeptide family. SP is one of five members of the tachykinin family that includes neurokinin A, neuropeptide K, neuropeptide γ, and neurokinin B in addition to SP. They are produced from a protein precursor after differential splicing of the prepro-Tachykinin A gene (Helke et al. 1990. FASEB Journal 4(6):1606-15). SP plays a role in nociception, inflammation, plasma extravasation, platelet and leukocyte aggregation in post-capillary venules, and leukocyte chemotactic migration through vessel walls (Otsuka M, Yoshioka K. Neurotransmitter functions of mammalian tachykinins. Physiol Rev. 1993 April; 73(2):229-308)

In the peripheral system, SP may regulate cardiovascular and renal function upon being released from sensory nerves innervating these organs/tissues (Wimalawansa S J. 1996. Endocr Rev 17:533-585)

Circulating Substance P was shown to be is increased in decompensated patients with liver cirrhosis and was inversely correlated with urinary sodium excretion and glomerular filtration rate (GFR) (Fernández-Rodriguez et al. 1995. Hepatology 21(1): 35-40).

The fasting SP plasma levels, measured by radioimmunoassay in stable patients with chronic renal failure receiving regular hemodialysis treatment were increased compared to healthy controls, concluding that elevated levels of gastrointestinal peptides (including SP) in patients with chronic renal failure may contribute to uremic gastrointestinal symptoms and dysfunctions (Hegbrant et al. 1991. Scand J Gastroenterol 26(6): 0.599-604; Hegbrant et al. 1992. Scand J Urol Nephrol 26(2): 169-76).

Pro-substance P (ProSP) levels were measured in patients with acute myocardial infarction (AMI) (Ng. et al 2014. JACC 64(16): 1698-1707, were highest on the first 2 days after admission and significantly negatively correlated to estimated glomerular filtration rate (eGFR). In this study proSP was most strongly correlated with renal function and may therefore closely reflect patients renal function at AMI presentation.

Investigations in man have been hampered by the very short half-life of SP (12 min) (Conlon and Sheehan, Regul. Pept. 1983: 7:335-345). The recent development of an assay for stable PTA (N-terminal pro-substance P; previously termed also N-terminal Pro-Tachykinin A or NT-PTA) which is a surrogate for labile SP (Ernst et al. Peptides 2008; 29: 1201-1206), has enabled studies on the role of this tachykinin system in human disease.

A subject of the present invention was also the provision of the prognostic and diagnostic power of PTA or fragments thereof for the diagnosis of kidney dysfunction and the prognostic value in diseased subjects.

Surprisingly, it has been shown that PTA or fragments are powerful and highly significant biomarker for kidney, its function, dysfunction, risk of death or adverse events or monitoring the success of a therapy or intervention or predicting the incidence of (chronic) kidney disease. Moreover, the measurement of PTA or fragments thereof can be used for the monitoring and/or decision for continuation and/or withdrawal of medications that are potentially harmful to the kidneys (nephrotoxic), e.g. antibiotics (for example vancomycin, gentamicin), analgesics, non-steroidal anti-inflammatory drugs (NSAID) (for example ibuprofen, naproxen), diuretics, proton pump inhibitors, chemotherapeutics (for example cisplatin), contrast dyes, cardiovascular agents like ACE-inhibitors or statins, anti-depressants and antihistamines (for reference see Naughton 2008. Am Fam Physician. 2008; 78(6):743-750, Table 1).

Subject matter of the present invention is method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or (e) predicting the incidence of (chronic) kidney disease intervention comprising:
determining the level of immunoreactive analyte by using at least one binder that binds to a region within the amino acid sequence of Pro-Tachykinin A (PTA) in a bodily fluid obtained from said subject; and
(a) correlating said level of immunoreactive analyte with kidney function in a subject, or
(b) correlating said level of immunoreactive analyte with kidney dysfunction wherein an elevated level above a certain threshold is predictive or diagnostic for kidney dysfunction in said subject, or
(c) correlating said level of immunoreactive analyte with said risk of an adverse event in a diseased subject, wherein an elevated level above a certain threshold is predictive for an enhanced risk of said adverse events, or
(d) correlating said level of immunoreactive analyte with success of a therapy or intervention in a diseased subject, wherein a level below a certain threshold is predictive for a success of therapy or intervention, or
(e) predicting the incidence of (chronic) kidney disease.

In a more specific embodiment subject matter of the present invention is a method for (a) diagnosing or monitoring kidney function in a subject or (b) diagnosing kidney dysfunction in a subject or (c) predicting the risk of death or an adverse event in a diseased subject, wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or (d) predicting or monitoring the success of a therapy or intervention or (e) predicting incidence of (chronic) kidney disease comprising:
determining the level of Pro-Tachykinin A or fragments thereof of at least 5 amino acids in a bodily fluid obtained from said subject; and
correlating said level of Pro-Tachykinin A or fragments thereof with kidney function in a subject, or
correlating said level of Pro-Tachykinin A or fragments thereof with kidney dysfunction wherein an elevated level above a certain threshold is predictive or diagnostic for kidney dysfunction in said subject, or
correlating said level of Pro-Tachykinin A or fragments thereof with a risk of death or an adverse event in a diseased subject, wherein an elevated level above a certain threshold is predictive for an enhanced risk of death or adverse events and wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or
correlating said level of Pro-Tachykinin A or fragments thereof with success of a therapy or intervention in a diseased subject, wherein a level below a certain threshold is predictive for a success of therapy or intervention, wherein said therapy or intervention is selected from the group comprising renal replacement therapy, and treatment with hyaluronic acid in patients having received renal replacement therapy, or
correlating said level of Pro-Tachykinin A or fragments thereof with the prediction or monitoring of the success of therapy or intervention comprising predicting or monitoring the recovery of renal function in patients with impaired renal function prior to and after renal replacement therapy, pharmaceutical interventions and/or adaption or withdrawal of nephrotoxic medications, or
correlating said level of Pro-Tachykinin A or fragments thereof with the prediction of incidence of (chronic) kidney disease.

The term “subject” as used herein refers to a living human or non-human organism. Preferably herein the subject is a human subject. The subject may be healthy or diseased if not stated otherwise.

The term “elevated level” means a level above a certain threshold level.

PTA and fragments thereof are early biomarker(s) for kidney, its function, dysfunction, risk of death or adverse events, monitoring the success of a therapy or intervention or predicting the incidence of (chronic) kidney disease. In this context PTA may be used as early surrogate for creatinine.

The term “early” as used herein means that the level of PTA and fragments thereof are elevated before elevations of creatinine are detectable. Elevations of PTA and fragments thereof may occur minutes, preferably hours, more preferably days before the creatinine levels are elevated. The term “early” as used herein may also mean within 24 hours after kidney function has changed or after the respective kidney event or an adverse event of kidney function.

Predicting or monitoring the success of a therapy or intervention may be e.g. the prediction or monitoring of success of renal replacement therapy using measurement of Pro-Tachykinin A or fragments thereof of at least 5 amino acids.

Predicting or monitoring the success of a therapy or intervention may be e.g. the prediction or monitoring of success of treatment with hyaluronic acid in patients having received renal replacement therapy using measurement of Pro-Tachykinin A or fragments thereof of at least 5 amino acids.

Predicting or monitoring the success of a therapy or intervention may be e.g. the prediction or monitoring recovery of renal function in patients with impaired renal function prior to and after renal replacement therapy and/or pharmaceutical interventions using measurement of Pro-Tachykinin A or fragments thereof of at least 5 amino acids.

A bodily fluid may be selected from the group comprising blood, serum, plasma, urine, cerebrospinal liquid (CSF), and saliva.

Determination of Pro-Tachykinin A or fragments thereof exhibit kidney function in a subject. An increased concentration of Pro-Tachykinin A indicates a reduced kidney function. During follow up measurements, a relative change of Pro-Tachykinin A or fragments thereof correlates with the improvement (lowering Pro-Tachykinin A or fragments thereof) and with the worsening (increased Pro-Tachykinin A or fragments thereof) of the subjects kidney function.

Pro-Tachykinin A or fragments thereof are diagnostic for kidney dysfunction wherein an elevated level above a certain threshold is predictive or diagnostic for kidney dysfunction in said subject. During follow up measurements, a relative change of Pro-Tachykinin A or fragments thereof correlates with the improvement (lowering Pro-Tachykinin A or fragments thereof) and with the worsening (increased Pro-Tachykinin A or fragments thereof) of the subjects kidney dysfunction.

Pro-Tachykinin A or fragments thereof are superior in comparison to other markers for kidney function/dysfunction diagnosis and follow up (NGAL, blood creatinine, creatinine clearance, Cystatin C, Urea). Superiority means higher specificity, higher sensitivity and better correlation to clinical endpoints. Pro-Tachykinin A or fragments thereof are in particular for the before-mentioned medical utilities in the patient group of Emergency Department all-corners.

Correlating said level of Pro-Tachykinin A or fragments thereof with a risk of death or an adverse event in a diseased subject, wherein an elevated level above a certain threshold is predictive for an enhanced risk of death or adverse events. Also in this aspect, Pro-Tachykinin A or fragments thereof are superior to above mentioned clinical markers.

The diseased person may suffer from a disease selected from chronic kidney disease (CKD), acute kidney disease (AKD) or acute kidney injury (AKI).

Conditions affecting kidney structure and function can be considered acute or chronic, depending on their duration.

AKD is characterized by structural kidney damage for <3 months and by functional criteria that are also found in AKI, or a GFR of <60 ml/min per 1.73 m² for <3 months, or a decrease in GFR by ≥35%, or an increase in serum creatinine (SCr) by >50% for <3 months (Kidney International Supplements, Vol. 2, Issue 1, March 2012, pp. 19-36).

AKI is one of a number of acute kidney diseases and disorders (AKD), and can occur with or without other acute or chronic kidney diseases and disorders.

AKI is defined as reduction in kidney function, including decreased GFR and kidney failure. The criteria for the diagnosis of AKI and the stage of severity of AKI are based on changes in SCr and urine output. In AKI no structural criteria are required (but may exist), but an increase in serum creatinine (SCr) by 50% within 7 days, or an increase by 0.3 mg/dl (26.5 μmol/l), or oliguria is found. AKD may occur in patients with trauma, stroke, sepsis, SIRS, septic shock, acute myocardial infarction (MI), post-MI, local and systemic bacterial and viral infections, autoimmune diseases, burned patients, surgery patients, cancer, liver diseases, lung diseases, as well as in patients receiving nephrotoxins such as cyclosporine, antibiotics including aminoglycosides and anticancer drugs such as cisplatin.

Kidney failure is a stage of AKI and is defined as a GFR<15 ml/min per 1.73 m2 body surface area, or requirement for renal replacement therapy (RRT).

CKD is characterized by a glomerular filtration rate (GFR) of <60 ml/min per 1.73 m² for >3 months and by kidney damage for >3 months (Kidney International Supplements, 2013; Vol. 3: 19-62).

The definitions of AKD, AKI and CKD (according to KDIGO Clinical Practice Guideline for Acute Kidney Injury 2012 Vol 2 (1)) are summarized in Table 1.

TABLE 1
Definition of AKI, AKD and CKD

| | Functional criteria | Structural criteria |
|---|---|---|
| AKI | Increase in SCr by 50% within 7 days, OR | No criteria |
| | Increase in SCr by 0.3 mg/dl (26.5 μmol/l) | |
| | within 2 days, OR | |
| | Oliguria | |
| AKD | AKI, OR | Kidney damage |
| | GFR <60 ml/min per 1.73 m² for | for >3 months |
| | <3 months, OR Decrease in GFR | |
| | by >35% or increase in SCr by | |
| | >50% for <3 months | |
| CKD | GFR <60 ml/min per 1.73 m² for | Kidney damage |
| | >3 months | for >3 months |
| NKD | GFR ≥60 ml/min per 1.73 m² | No damage |
| | Stable SCr | |

NKD = no kidney disease

The acronym RIFLE stands for the increasing severity classes Risk, Injury, and Failure; and the two outcome classes, Loss and End-Stage Renal Disease (ESRD). The three severity grades are defined on the basis of the changes in SCr or urine output where the worst of each criterion is used. The two outcome criteria, Loss and ESRD, are defined by the duration of loss of kidney function.

The Acute Kidney Injury Network (AKIN), endorsed the RIFLE criteria with a small modification to include small changes in SCr (≥0.3 mg/dl or ≥26.5 μmol/1) when they occur within a 48-hour period.

A comparison of RIFLE and AKIN criteria for classification of AKI (according to KDIGO Clinical Practice Guideline for Acute Kidney Injury 2012 Vol 2 (1)) is presented in Table 2.

TABLE 2
comparison of RIFLE and AKIN criteria

| | Urine | | |
|---|---|---|---|
| | output | | |
| AKI staging (AKIN) | (common | RIFLE | |
| Serum creatinine | to both) | Class | Serum creatinine or GFR |
| Stage 1 Increase of | Less than | Risk | Increase in serum |
| more than or equal to | 0.5 ml/kg/h | | creatinine ×1.5 |
| 0.3 mg/dl (>26.5 | for more | | or GFR |
| μmol/l) or increase | than | | decrease >25% |
| to more than or | 6 hours | | |
| equal to 150% to 200% | | | |
| (1.5- to 2-fold) from | | | |
| baseline | | | |
| Stage 2 Increased to | Less than | Injury | Serum creatinine ×2 |
| more than 200% to | 0.5 ml/kg | | or GFR decreased |
| 300% (>2- to 3-fold) | per hour | | >50% |
| from baseline | for more | | |
| | than | | |
| | 12 hours | | |
| Stage 3 Increased to | Less than | Failure | Serum creatinine ×3, |
| more than 300% (>3- | 0.3 ml/kg/h | | or serum creatinine |
| fold) from baseline, | for 24 | | >4 mg/dl (>354 μmol/l) |
| or more than or equal | hours or | | with an acute |
| to 4.0 mg/dl (354 | anuria for | | rise >0.5 mg/dl |
| μmol/l) with an acute | 12 hours | | (>44 μmol/l) |
| increase of at least 0.5 | | | or GFR |
| mg/dl (44 μmol/l) or | | | decreased >75% |
| on RRT | | | |
| | | Loss | Persistent acute renal |
| | | | failure = complete |
| | | | loss of kidney |
| | | | function >4 weeks |
| | | End- | ESRD >3 months |
| | | stage | |
| | | kidney | |
| | | disease | |

Risk according to the present invention correlates with the risk as defined by the RIFLE criteria (Hoste et al. 2006. Critical Care 10: R73).

An adverse event may be selected from the group comprising worsening of kidney dysfunction including kidney failure, loss of kidney function and end-stage kidney disease (according to the RIFLE criteria, Hoste et al. 2006. Critical Care 10: R73).

The therapy or intervention supporting or replacing kidney function may comprise various methods of renal replacement therapy including but not limited to hemodialysis, peritoneal dialysis, hemofiltration and renal transplantation.

The therapy or intervention supporting or replacing kidney function may also comprise pharmaceutical interventions, kidney-supporting measures as well as adaption and/or withdrawal of nephrotoxic medications, antibiotics and diuretica.

In the context of the present invention, an adverse event is selected from the group comprising worsening of kidney dysfunction including kidney failure, loss of kidney function and end-stage kidney disease or death due to kidney dysfunction including kidney failure, loss of kidney function and end-stage kidney disease. In the context of predicting or monitoring the success of a therapy or intervention, said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions.

Throughout the specification the term Pro-Tachykinin and Pro-Tachykinin A (PTA) are used synonymously. The term includes all splice variants of Pro-Tachykinin A, namely αPTA, βPTA, γPTA, and δPTA. Throughout the specification it should be understood that the term fragments of Pro-Tachykinin A also include Substance P and Neurokinin A, Neuropeptide K, Neuropeptide γ, and Neurokinin B if not stated otherwise.

The term “determining the level of Pro-Tachykinin, its splice variants or fragments thereof of at least 5 amino acids including Substance P and Neurokinin” means that usually the immunoreactivity towards a region within the before mentioned molecules is determined. This means that it is not necessary that a certain fragment is measured selectively. It is understood that a binder which is used for the determination of the level of Pro-Tachykinin or fragments thereof of at least 5 amino acids including Substance P and Neurokinin binds to any fragment that comprises the region of binding of said binder. Said binder may be an antibody or antibody fragment or a non-IgG Scaffold.

Subject matter according to the present invention is a method wherein the level of Pro-Tachykinin A or fragments thereof of at least 5 amino acids is determined by using a binder to Pro-Tachykinin A or fragments thereof of at least 5 amino acids.

In one embodiment of the invention said binder is selected from the group comprising an antibody, an antibody fragment or a non-Ig-Scaffold binding to Pro-Tachykinin A or fragments thereof of at least 5 amino acids.

Alternative splicing of the PTA gene transcript generates four different PTA-mRNA molecules designated as αPTA, βPTA, γPTA, and δPTA, respectively (Harmar et al. 1990. FEBS Lett 275:22-4; Kawaguchi et al. 1986. Biochem Biophys Res Commun 139: 1040-6; Naiwa et al. 1984. Nature 312:729-34), that differ in their exon combinations. All seven exons are solely contained in beta-PTA mRNA. However, the first three exons encoding for SP and a common N-terminal region consisting of 37 amino acids (SEQ ID NO. 5), are present in all PTA precursor molecules.

Alternative splicing gives the following Pro-Tachykinin A sequences:

(Isoform αPTA)
SEQ ID NO. 1
EEIGANDDLNYWSDWYDSDQIKEELPEPFEHLLQRIARRPKPQQFFGLMGK
RDADSSIEKQVALLKALYGHGQISHKMAYERSAMQNYERRR
(Isoform βPTA)
SEQ ID NO. 2
EEIGANDDLNYWSDWYDSDQIKEELPEPFEHLLQRIARRPKPQQFFGLMGK
RDADSSIEKQVALLKALYGHGQISHKRHKTDSFVGLMGKRALNSVAYERSA
MQNYERRR
(Isoform γPTA)
SEQ ID NO. 3
EEIGANDDLNYWSDWYDSDQIKEELPEPFEHLLQRIARRPKPQQFFGLMGK
RDAGHGQISHKRHKTDSFVGLMGKRALNSVAYERSAMQNYERRRSEQ
(Isoform δPTA)
SEQ ID NO. 4
EEIGANDDLNYWSDWYDSDQIKEELPEPFEHLLQRIARRPKPQQFPGLMGK
RDAGHGQISHKMAYERSAMQNYERRR

Fragments of Pro-Tachykinin A that may be determined in a bodily fluid may be e.g. selected from the group of the following fragments:

(Pro-Tachykinin A 1-37, P37, NT-PTA)
SEQ ID NO. 5
EEIGANDDLNYWSDWYDSDQIKEELPEPFEHLLQRIA
(Substance P)
SEQ ID NO. 6
RPKPQQFFGLM(—NH₂)
(Neuropeptide K)
SEQ ID NO. 7
DADSSIEKQVALLKALYGHGQISHKRHKTDSFVGLM (—NH₂)
(Neuropeptide Gamma)
SEQ ID NO. 8
GHGQISHKRHKTDSFVGLM (—NH₂)
(Neurokinin B)
SEQ ID NO. 9
HKTDSFVGLM(—NH₂)
(C-terminal flanking peptide, PTA 92-107)
SEQ ID NO. 10
ALNSVAYERSAMQNYE
(PTA 3-22)
SEQ ID NO. 11
GANDDLNYWSDWYDSDQIK
(PTA 21-36)
SEQ ID NO. 12
IKEELPEPFEHLLQRI

Determining the level of Pro-Tachykinin A or fragments thereof may mean that the immunoreactivity towards PTA or fragments thereof including Substance P and Neurokinin is determined. A binder used for determination of PTA or fragments thereof depending of the region of binding may bind to more than one of the above displayed molecules. This is clear to a person skilled in the art.

In a more specific embodiment of the invention fragments of PTA may be selected from SEQ ID NO. 5, SEQ ID NO. 10, SEQ ID NO. 11 and SEQ ID NO. 12.

In a more specific embodiment of the method according to the present invention the level of P37 (also termed PTA 1-37 or NT-PTA, SEQ ID NO. 5, EEIGANDDLNYWSDWYDSDQIKEELPEPFEHLLQRIA) is determined. In an even more specific embodiment according to the present invention at least one or two binders are used that bind to PTA 1-37 (NT-PTA), SEQ ID NO. 5, EEIGANDDLNYWSDWYDSDQIKEELPEPFEHLLQRIA, in case of more than one binder they bind preferably to two different regions within PTA 1-37 (NT-PTA), SEQ ID NO. 5, EEIGANDDLNYWSDWYDSDQIKEELPEPFEHLLQRIA. Said binder(s) may preferably be an antibody or a binding fragment thereof.

In an even more specific embodiment binder(s) are used for the determination of PTA, its variants and fragments that bind to one or both, respectively, of the following regions within PTA 1-37 (NT-PTA): PTA 3-22 (GANDDLNYWSDWYDSDQIK, which is SEQ ID NO. 11) and PTA 21-36 (IKEELPEPFEHLLQRI, which is SEQ ID NO. 12).

Thus, according to the present invention the level of immunoreactive analyte by using at least one binder that binds to a region within the amino acid sequence of any of the above peptide and peptide fragments, (i.e. Pro-Tachykinin A (PTA) and fragments according to any of the sequences 1 to 12), is determined in a bodily fluid obtained from said subject; and correlated to the specific embodiments of clinical relevance.

In a more specific embodiment of the method according to the present invention the level of PTA 1-37 is determined (SEQ ID NO. 5: NT-PTA).

In a more specific embodiment the level of immunoreactive analyte by using at least one binder that binds to NT-PTA is determined and is correlated to the above mentioned embodiments according to the invention to the specific embodiments of clinical relevance, e.g.
correlating said level of immunoreactive analyte with kidney function in a subject or
(a) correlating said level of immunoreactive analyte with kidney dysfunction wherein an elevated level above a certain threshold is predictive or diagnostic for kidney dysfunction in said subject, or
(b) correlating said level of immunoreactive analyte with said risk of an adverse event in a diseased subject, wherein an elevated level above a certain threshold is predictive for an enhanced risk of said adverse events, or
(c) correlating said level of immunoreactive analyte with success of a therapy or intervention in a diseased subject, wherein a level below a certain threshold is predictive for a success of therapy or intervention, or
(d) predicting the incidence of (chronic) kidney disease.

In a more specific embodiment the level of immunoreactive analyte by using at least one binder that binds to NT-PTA is determined and is correlated to the above-mentioned embodiments according to the invention to the specific embodiments of clinical relevance, e.g.
correlating said level of immunoreactive analyte with kidney function in a subject, or
correlating said level of immunoreactive analyte with kidney function in a subject, or
correlating said level of immunoreactive analyte with kidney dysfunction wherein an elevated level above a certain threshold is predictive or diagnostic for kidney dysfunction in said subject, or
correlating said level of of immunoreactive analyte with a risk of death or an adverse event in a diseased subject, wherein an elevated level above a certain threshold is predictive for an enhanced risk of death or adverse events and wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or
correlating said level of immunoreactive analyte with success of a therapy or intervention in a diseased subject, wherein a level below a certain threshold is predictive for a success of therapy or intervention, wherein said therapy or intervention is selected from the group comprising renal replacement therapy, and treatment with hyaluronic acid in patients having received renal replacement therapy, or
correlating said level of immunoreactive analyte with the prediction or monitoring of the success of therapy or intervention comprising predicting or monitoring the recovery of renal function in patients with impaired renal function prior to and after renal replacement therapy, pharmaceutical interventions and/or adaption or withdrawal of nephrotoxic medications, or
correlating said level of immunoreactive analyte with the prediction of incidence of (chronic) kidney disease.

Alternatively, the level of any of the above analytes may be determined by other analytical methods e.g. mass spectroscopy.

Thus, subject matter of the present invention is method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting the incidence of (chronic) kidney disease comprising:
determining the level of immunoreactive analyte by using at least one binder that binds to a region within the amino acid sequence of a peptide selected from the group comprising the peptides and fragments of SEQ ID NO. 1 to 12 in a bodily fluid obtained from said subject; and
   correlating said level of Pro-Tachykinin or fragments thereof with kidney function in a subject, or
   correlating said level of Pro-Tachykinin A or fragments thereof with kidney dysfunction wherein an elevated level above a certain threshold is predictive or diagnostic for kidney dysfunction in said subject, or
   correlating said level of Pro-Tachykinin A or fragments thereof with said risk of an adverse event in a diseased subject, wherein an elevated level above a certain threshold is predictive for an enhanced risk of said adverse events, or
   correlating said level of Pro-Tachykinin A or fragments thereof with success of a therapy or intervention in a diseased subject, wherein a level below a certain threshold is predictive for a success of therapy or intervention, or
   predicting the incidence of (chronic) kidney disease.

In a more specific embodiment subject matter of the present invention is a method for (a) diagnosing or monitoring kidney function in a subject or (b) diagnosing kidney dysfunction in a subject or (c) predicting the risk of death or an adverse event in a diseased subject, wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or (d) predicting or monitoring the success of a therapy or intervention or (e) predicting incidence of (chronic) kidney disease comprising:
determining the level of of immunoreactive analyte in a bodily fluid obtained from said subject; and
correlating said level of immunoreactive analyte with kidney function in a subject, or
correlating said level of immunoreactive analyte with kidney function in a subject, or
correlating said level of immunoreactive analyte with kidney dysfunction wherein an elevated level above a certain threshold is predictive or diagnostic for kidney dysfunction in said subject, or
correlating said level of of immunoreactive analyte with a risk of death or an adverse event in a diseased subject, wherein an elevated level above a certain threshold is predictive for an enhanced risk of death or adverse events and wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or
correlating said level of immunoreactive analyte with success of a therapy or intervention in a diseased subject, wherein a level below a certain threshold is predictive for a success of therapy or intervention, wherein said therapy or intervention is selected from the group comprising renal replacement therapy, and treatment with hyaluronic acid in patients having received renal replacement therapy, or
correlating said level of immunoreactive analyte with the prediction or monitoring of the success of therapy or intervention comprising predicting or monitoring the recovery of renal function in patients with impaired renal function prior to and after renal replacement therapy, pharmaceutical interventions and/or adaption or withdrawal of nephrotoxic medications, or
correlating said level of immunoreactive analyte with the prediction of incidence of (chronic) kidney disease.

In one embodiment of the invention said binder is selected from the group comprising an antibody, an antibody fragment, a non-Ig-Scaffold or aptamers binding to Pro-Tachykinin A or fragments thereof of at least 5 amino acids.

In a more specific embodiment the level of immunoreactive analyte by using at least one binder that binds to a region within the amino acid sequence of Pro-Tachykinin 1-37, N-terminal Pro-Tachykinin A fragment, NT-PTA (SEQ ID NO. 5) in a bodily fluid obtained from said subject.

In a specific embodiment the level of Pro-Tachykinin A or fragments thereof are measured with an immunoassay using antibodies or fragments of antibodies binding to Pro-Tachykinin A or fragments thereof. An immunoassay that may be useful for determining the level of Pro-Tachykinin A or fragments thereof of at least 5 amino acids may comprise the steps as outlined in Example 1. All thresholds and values have to be seen in correlation to the test and the calibration used according to Example 1. A person skilled in the art may know that the absolute value of a threshold might be influenced by the calibration used. This means that all values and thresholds given herein are to be understood in context of the calibration used in herein (Example 1).

According to the invention the diagnostic binder to Pro-Tachykinin A is selected from the group consisting of antibodies e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins.

In a specific embodiment the level of Pro-Tachykinin A or fragments thereof are measured with an assay using binders selected from the group comprising an antibody, an antibody fragment aptamers, non-Ig scaffolds as described in greater detail below binding to Pro-Tachykinin A or fragments thereof.

Binder that may be used for determining the level of Pro-Tachykinin A or fragments thereof exhibit an affinity constant to Pro-Tachykinin A or fragments thereof of at least 10⁷ M⁻¹, preferred 10⁸ M⁻¹, preferred affinity constant is greater than 10⁹ M⁻¹, most preferred greater than 10¹⁰ M⁻¹. A person skilled in the art knows that it may be considered to compensate lower affinity by applying a higher dose of compounds and this measure would not lead out-of-the-scope of the invention. Binding affinity may be determined using the Biacore method, offered as service analysis e.g. at Biaffin, Kassel, Germany (http://www.biaffin.com/de/).

To determine the affinity of the antibodies, the kinetics of binding of PTA splice variants or fragments thereof to immobilized antibody was determined by means of label-free surface plasmon resonance using a Biacore 2000 system (GE Healthcare Europe GmbH, Freiburg, Germany). Reversible immobilization of the antibodies was performed using an anti-mouse Fc antibody covalently coupled in high density to a CM5 sensor surface according to the manufacturer's instructions (mouse antibody capture kit; GE Healthcare). (Lorenz et al., “Functional Antibodies Targeting IsaA of Staphylococcus aureus Augment Host Immune Response and Open New Perspectives for Antibacterial Therapy”; Antimicrob Agents Chemother. 2011 January; 55(1): 165-173)

A human PTA-control sample is available by ICI-Diagnostics, Berlin, Germany http://www.ici-diagnostics.com/. The assay may also be calibrated by synthetic (for our experiments we used synthetic P37, SEQ ID NO. 5) or recombinant PTA splice variants or fragments thereof.

In addition to antibodies other biopolymer scaffolds are well known in the art to complex a target molecule and have been used for the generation of highly target specific biopolymers. Examples are aptamers, spiegelmers, anticalins and conotoxins. Non-Ig scaffolds may be protein scaffolds and may be used as antibody mimics as they are capable to bind to ligands or antigenes. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds (e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

The threshold for diagnosing kidney disease/dysfunction or for determining the risk of death or an adverse event or predicting or monitoring the success of a therapy or intervention or predicting incidence of (chronic) kidney disease may be the upper normal range (99 percentile, 107 pmol NT-PTA/L, more preferred 100 pmol/L, even more preferred 80 pmol/L). A threshold range is useful between 80 and 100 pmol NT-PTA/L.

In one specific embodiment the level of Pro-Tachykinin A is measured with an immunoassay and said binder is an antibody, or an antibody fragment binding to Pro-Tachykinin A or fragments thereof of at least 5 amino acids.

In one specific embodiment the assay used comprises two binders that bind to two different regions within the region of Pro-Tachykinin A that is amino acid 3-22 (sequence, SEQ ID NO. 11) and amino acid 21-36 (sequence, SEQ ID NO. 12) wherein each of said regions comprises at least 4 or 5 amino acids.

In one embodiment of the assays for determining Pro-Tachykinin A or Pro-Tachykinin A fragments in a sample according to the present invention the assay sensitivity of said assay is able to quantify the Pro-Tachykinin A or Pro-Tachykinin A fragments of healthy subjects and is <20 pmol/, preferably <10 pmol/L and more preferably <5 pmol/L.

Subject matter of the present invention is the use of at least one binder that binds to a region within the amino acid sequence of a peptide selected from the group comprising the peptides and fragments of SEQ ID NO. 1 to 12 in a bodily fluid obtained from said subject in a method a for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting the incidence of (chronic) kidney disease. In one embodiment of the invention said binder is selected from the group comprising an antibody, an antibody fragment or a non-Ig-Scaffold binding to Pro-Tachykinin A or fragments thereof of at least 5 amino acids. In a specific embodiment said at least one binder binds to a region with the sequences selected from the group comprising SEQ ID NO. 1, 2, 3, 4, 5, 6, 7, 8, 9 10, 11 and 12. In a specific embodiment said binder do not bind to SEQ ID NO. 6, 7, 8 and 9. In a specific embodiment said at least one binder binds to a region with the sequences selected from the group comprising SEQ ID No. 1, 2, 3, 4, 5, 11 and 12. In another specific embodiment said at least one binder binds to a region with the sequences selected from the group comprising SEQ ID No. 5, 11 and 12. In another very specific embodiment said binder bind to Pro-Tachykinin A 1-37, N-terminal Pro-Tachykinin A fragment, NT-PTA (SEQ ID NO. 5).

In a more specific embodiment the at least one binder binds to a region within the amino acid sequence of Pro-Tachykinin A 1-37, N-terminal Pro-Tachykinin A fragment, NT-PTA (SEQ ID NO. 5) in a bodily fluid obtained from said subject, more specifically to amino acid 3-22 (GANDDLNYWSDWYDSDQIK, SEQ ID NO. 11) and/or amino acid 21-36 (IKEELPEPFEHLLQRI, SEQ ID NO. 12) wherein each of said regions comprises at least 4 or 5 amino acids.

Thus, according to the present methods the level of immunoreactivity of the above binder is determined in a bodily fluid obtained from said subject. Level of immunoreactivity means the concentration of an analyte determined quantitatively, semi-quantitatively or qualitatively by a binding reaction of a binder to such analyte, where preferably the binder has an affinity constant for binding to the analyte of at least 10⁸ M⁻¹, and the binder may be an antibody or an antibody fragment or an non-IgG scaffold, and the binding reaction is an immunoassay.

The present methods using Pro-Tachykinin A and fragments thereof, especially NT-PTA, are far superior over the methods and biomarkers used according to the prior art for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting the incidence of (chronic) kidney disease. Similar to Proenkephalin (PENK), PTA and fragments thereof as biomarker for the before mentioned uses is an inflammation independent marker. That is an important feature as most of the known kidney biomarker like NGAL and KIM are inflammation dependent, meaning if the subject has an inflammation, e.g. in sepsis, the elevation of NGAL or KIM may be either due to inflammation or to kidney function/dysfunction. Thus, no differential diagnosis may be conducted, at least not by using a simple cut-off value (meaning one (1) cut-off value), which is independent from the particular patient population investigated. For NGAL and KIM each and every patient has an “individual” threshold for kidney function/dysfunction depending on the inflammation status of said subject which makes clinical application of these kidney markers difficult in some diseases and impossible in others. In contrast thereto, one single threshold that is independent of the inflammation status of the subject may be used according to the present methods for all subjects. This makes the present methods suitable for clinical routine in contrast to the before-mentioned inflammation-dependent markers.

PTA and fragments thereof as biomarker in the methods of the present invention, especially NT-PTA reflects “real” kidney function in contrast to NGAL and KIM, they reflect kidney damage and inflammation.

Thus, subject matter of the present invention is method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting the incidence of (chronic) kidney disease with the before mentioned steps and features wherein an inflammation status independent threshold is used.

Another advantage of the above methods and the use of PTA and fragments as biomarker in the methods for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention or (e) predicting the incidence of (chronic) kidney disease is that PTA and fragments as biomarker are very early biomarker for kidney function, kidney dysfunction, risk of an adverse event, success of a therapy or intervention or predicting the incidence of (chronic) kidney disease. Very early means e.g. earlier than creatinine, earlier than NGAL.

One clear indication of the superiority of PTA over creatinine comes from an analysis of the association of the respective concentrations determined in critically ill patients on the day of admission with their 7 day mortality rate (Example 6): PTA concentrations of survivors differ significantly from non-survivors, whereas this is not the case for creatinine clearance. Mortality in such patient population is mainly driven by loss of kidney function. Thus, the significant and much stronger association of PTA with mortality than of creatinine clearance supports the superiority of PTA over creatinine clearance as kidney dysfunction marker.

Subject of the present invention is also a method for (a) diagnosing or monitoring kidney function in subject or (b) diagnosing kidney dysfunction in a subject or (c) predicting or monitoring the risk of an adverse events in a diseased subject wherein said adverse event is selected from the group comprising worsening of kidney dysfunction including kidney failure, loss of kidney function and end-stage kidney disease or death due to kidney dysfunction including kidney failure, loss of kidney function and end-stage kidney disease or (d) predicting or monitoring the success of a therapy or intervention supporting or replacing kidney function comprising various methods of renal replacement therapy including but not limited to hemodialysis, peritoneal dialysis, hemofiltration and renal transplantation or (e) predicting the incidence of (chronic) kidney disease according to any of the preceding embodiments, wherein the level of Pro-Tachykinin A or fragments thereof of at least 5 amino acids in a bodily fluid obtained from said subject either alone or in conjunction with other prognostically useful laboratory or clinical parameters is used which may be selected from the following alternatives:
Comparison with the median of the level of Pro-Tachykinin A or fragments thereof of at least 5 amino acids in a bodily fluid obtained from said subject in an ensemble of pre-determined samples in a population of “healthy” or “apparently healthy” subjects,
Comparison with a quantile of the level of Pro-Tachykinin A or fragments thereof of at least 5 amino acids in a bodily fluid obtained from said subject in an ensemble of pre-determined samples in a population of “healthy” or “apparently healthy” subjects,
Calculation based on Cox Proportional Hazards analysis or by using Risk index calculations such as the NRI (Net Reclassification Index) or the IDI (Integrated Discrimination Index).

Said additionally at least one clinical parameter may be determined selected from the group comprising: age, blood urea nitrogen (BUN), neutrophil gelatinase-associated lipocalin (NGAL), proenkephalin (PENK), Cystatin C, Creatinine Clearance, Creatinine, Urea, Apache Score, systolic blood pressure and/or diastolic blood pressure (SBP and/or DBP), antihypertensive treatment (AHT), body mass index (BMI), body fat mass, body lean mass, waist circumference, waist-hip-ratio, current smoker, diabetes heredity, cardiovascular disease (CVD), total cholesterol, triglyceride, low-density-lipocholesterol (LDL-C), high-density-lipocholesterol (HDL-C), whole blood or plasma glucose, plasma insulin, HOMA (Insulin (μU/ml)×Glucose (mmol/l)/22.5), and/or HbA_{1c} (%), optionally further comprising determining the status of genetic markers.

In addition to the determination of the level of PTA, its splice variants or fragments thereof of at least 5 amino acids including Substance P and Neurokinin in a bodily fluid obtained from said subject, Pro-Enkephalin (PENK) or fragments of at least 5 amino acids thereof may be measured in a bodily fluid obtained from said subject. It has to be understood that in addition to the determination of the level of PTA, its splice variants or fragments thereof of at least 5 amino acids Pro-Enkephalin (PENK) or fragments of at least 5 amino acids thereof may be measured in a bodily fluid obtained from said subject. This means that the level of either PTA alone or in combination with PENK is measured and correlated with said risk.

In a more specific embodiment of the method according to the present invention the level Pro-Enkephalin (PENK) or fragments of at least 5 amino acids thereof is determined in addition to the determination of the level of PTA, its splice variants or fragments thereof.

Thus, subject matter of the invention is also a method for diagnosing or monitoring kidney function in subject or diagnosing kidney dysfunction in a subject or predicting the risk of death or adverse events or predicting or monitoring the success of a therapy or intervention or predicting incidence of (chronic) kidney disease in a diseased subject comprising:
determining the level of Pro-Tachykinin A or fragments thereof of at least 5 amino acids in a bodily fluid obtained from said subject; and
determining the level of Pro-Enkephalin or fragments thereof of at least 5 amino acids in a bodily fluid obtained from said subject; and

correlating said level of PTA, its splice variants or fragments thereof and the level of Pro-Enkephalin or fragments thereof of at least 5 amino acids with kidney function in a subject, or

correlating said level of PTA, its splice variants or fragments thereof and the level of Pro-Enkephalin or fragments thereof of at least 5 amino acids with kidney dysfunction wherein an elevated level above a certain threshold is predictive or diagnostic for kidney dysfunction in said subject, or

correlating said level of PTA, its splice variants or fragments thereof and the level of Pro-Enkephalin or fragments thereof of at least 5 amino acids with a risk of death or an adverse event in a diseased subject, wherein an elevated level above a certain threshold is predictive for an enhanced risk of death or adverse events, or correlating said level of PTA, its splice variants or fragments thereof and the level of Pro-Enkephalin or fragments thereof of at least 5 amino acids with the success of a therapy or intervention in a diseased subject, wherein a level below a certain threshold is predictive for a success of therapy or intervention, or

correlating said level of PTA, its splice variants or fragments thereof and the level of Pro-Enkephalin or fragments thereof of at least 5 amino acids with the prediction of incidence of (chronic) kidney disease wherein a level below a certain threshold is predictive for a success of therapy or intervention.

Pro-Enkephalin and fragments may have the following sequence:

(Pro-Enkephalin (1-243)
SEQ ID NO. 13
ECSQDCATCSYRLVRPADINFLACVMECEGKLPSLKIWETCKELLQLSKPE
LPQDGTSTLRENSKPEESHLLAKRYGGFMKRYGGFMKKMDELYPMEPEEEA
NGSEILAKRYGGFMKKDAEEDDSLANSSDLLKELLETGDNRERSHHQDGSD
NEEEVSKRYGGFMRGLKRSPQLEDEAKELQKRYGGFMRRVGRPEWWMDYQK
RYGGFLKRFAEALPSDEEGESYSKEVPEMEKRYGGFMRF

Fragments of Pro-Enkephalin that may be determined in a bodily fluid may be e.g. selected from the group of the following fragments:

(Syn-Enkephalin, Pro-Enkephalin 1-73)
SEQ ID NO. 14
ECSQDCATCSYRLVRPADINFLACVMECEGKLPSLKIWETCKELLQLSKPE
LPQDGTSTLRENSKPEESHLLA
(Met-Enkephalin)
SEQ ID NO. 15
YGGFM
(Leu-Enkephalin)
SEQ ID NO. 16
YGGFL
(Pro-Enkephalin 90-109)
SEQ ID NO. 17
MDELYPMEPEEEANGSEILA
(Pro-Enkephalin 119-159, Mid-regional Pro-
Enkephalin fragment, MR-PENK)
SEQ ID NO. 18
DAEEDDSLANSSDLLKELLETGDNRERSHHQDGSDNEEEVS
(Met-Enkephalin-Arg-Gly-Leu)
SEQ ID NO. 19
YGGFMRGL
(Pro-Enkephalin 172-183)
SEQ ID NO. 20
SPQLEDEAKELQ
(Pro-Enkephalin 193-203)
SEQ ID NO. 21
VGRPEWWMDYQ
(Pro-Enkephalin 213-234)
SEQ ID NO. 22
FAEALPSDEEGESYSKEVPEME
(Pro-Enkephalin 213-241)
SEQ ID NO. 23
FAEALPSDEEGESYSKEVPEMEKRYGGFM
(Met-Enkephalin-Arg-Phe)
SEQ ID NO. 24
YGGFMRF

Determining the level of Pro-Enkephalin including Leu-Enkephalin and Met-Enkephalin or fragments thereof may mean that the immunoreactivity towards Pro-Enkephalin or fragments thereof including Leu-Enkephalin and Met-Enkephalin is determined. A binder used for determination of Pro-Enkephalin including Leu-Enkephalin and Met-Enkephalin or fragments thereof depending of the region of binding may bind to more than one of the above displayed molecules. This is clear to a person skilled in the art.

In a more specific embodiment of the method according to the present invention the level of MR-PENK (SEQ ID NO. 18: (Pro-Enkephalin 119-159, Mid-regional Pro-Enkephalin-fragment, MR-PENK)), which is DAEEDDSLANSSDLLKELLETGDNRERSHHQDGSDNEEEVS, is determined.

In a specific embodiment the level of Pro-Enkephalin or fragments thereof is measured with an immunoassay using antibodies or fragments of antibodies binding to Pro-Enkephalin or fragments thereof (WO2014053501).

In one embodiment of the invention, said method is performed more than once in order to monitor the function or dysfunction or risk of said subject or in order to monitor the course of treatment of kidney and/or disease. In one specific embodiment said monitoring is performed in order to evaluate the response of said subject to preventive and/or therapeutic measures taken.

In one embodiment of the invention, the method is used in order to stratify said subjects into risk groups.

A variety of immunoassays are known and may be used for the assays and methods of the present invention, these include: radioimmunoassays (“RIA”), homogeneous enzyme-multiplied immunoassays (“EMIT”), enzyme linked immunoadsorbent assays (“ELISA”), apoenzyme reactivation immunoassay (“ARIS”), chemiluminescence- and fluorescence-immunoassays, Luminex-based bead arrays, protein microarray assays, and rapid test formats such as for instance immunochromatographic strip tests (“dipstick immunoassays”) and immuno-chromotography assays.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

In one embodiment of the invention it may be a so-called POC-test (point-of-care), that is a test technology which allows performing the test within less than 1 hour near the patient without the requirement of a fully automated assay system. One example for this technology is the immunochromatographic test technology.

In one embodiment of the invention at least one of said two binders is labeled in order to be detected.

In a preferred embodiment said label is selected from the group comprising chemiluminescent label, enzyme label, fluorescence label, radioiodine label.

The assays can be homogenous or heterogeneous assays, competitive and non-competitive assays. In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5-or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology 4th ed., executive editor, J. I. Kroschwitz; editor, M. Howe-Grant. John Wiley & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “binder molecules” are molecules which may be used to bind target molecules or molecules of interest, i.e. analytes (i.e. in the context of the present invention Pro-Tachyinin A and fragments thereof), from a sample. Binder molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

Chemiluminescent label may be acridinium ester label, steroid labels involving isoluminol labels and the like.

Enzyme labels may be lactate dehydrogenase (LDH), creatinekinase (CPK), alkaline phosphatase, aspartate aminotransferace (AST), alanine aminotransferace (ALT), acid phosphatase, glucose-6-phosphate dehydrogenase and so on.

In one embodiment of the invention at least one of said two binders is bound to a solid phase as magnetic particles, and polystyrene surfaces.

In one embodiment of the assays for determining Pro-Tachykinin A or Pro-Tachykinin A fragments in a sample according to the present invention such assay is a sandwich assay, preferably a fully automated assay. It may be an ELISA fully automated or manual. It may be a so-called POC-test (point-of-care). Examples of automated or fully automated assay comprise assays that may be used for one of the following systems: Roche Elecsys®, Abbott Architect®, Siemens Centauer®, Brahms Kryptor®, Biomerieux Vidas®, Alere Triage®. Examples of test formats are provided above.

In one embodiment of the assays for determining Pro-Tachykinin A or fragments in a sample according to the present invention at least one of said two binders is labeled in order to be detected. Examples of labels are provided above.

In one embodiment of the assays for determining Pro-Tachykinin A or fragments in a sample according to the present invention at least one of said two binders is bound to a solid phase. Examples of solid phases are provided above.

In one embodiment of the assays for determining Pro-Tachykinin A or fragments in a sample according to the present invention said label is selected from the group comprising chemiluminescent label, enzyme label, fluorescence label, radioiodine label. A further subject of the present invention is a kit comprising an assay according to the present invention wherein the components of said assay may be comprised in one or more container.

In one embodiment subject matter of the present invention is a point-of-care device for performing a method according to the invention wherein said point of care device comprises at least one antibody or antibody fragment directed to either amino acid 3-22 (GANDDLNYWSDWYDSDQIK, SEQ ID NO. 11) or amino acid 21-36 (IKEELPEPFEHLLQRI, SEQ ID NO. 12) wherein each of said regions comprises at least 4 or 5 amino acids.

In one embodiment subject matter of the present invention is a point-of-care device for performing a method according to the invention wherein said point of care device comprises at least two antibodies or antibody fragments directed to amino acid 3-22 (GANDDLNYWSDWYDSDQIK, SEQ ID NO. 11) and amino acid 21-36 (IKEELPEPFEHLLQRI, SEQ ID NO. 12), wherein each of said regions comprises at least 4 or 5 amino acids.

In one embodiment subject matter of the present invention is a kit or performing a method according to the invention wherein said point of care device comprises at least one antibody or antibody fragment directed to either amino acid 3-22 (GANDDLNYWSDWYDSDQIK, SEQ ID No. 11) or amino acid 21-36 (IKEELPEPFEHLLQRI, SEQ ID NO. 12) wherein each of said regions comprises at least 4 or 5 amino acids.

In one embodiment subject matter of the present invention is a kit for performing a method according to the invention, wherein said point of care device comprises at least two antibodies or antibody fragments directed to amino acid 3-22 (GANDDLNYWSDWYDSDQIK, SEQ ID NO. 11) and amino acid 21-36 (IKEELPEPFEHLLQRI, SEQ ID NO. 12), wherein each of said regions comprises at least 4 or 5 amino acids.

The following embodiments are subject of the present invention:
1. A method for (a) diagnosing or monitoring kidney function in a subject or (b) diagnosing kidney dysfunction in a subject or (c) predicting the risk of death or an adverse event in a diseased subject, wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or (d) predicting or monitoring the success of a therapy or intervention or (e) predicting incidence of (chronic) kidney disease comprising:
   determining the level of Pro-Tachykinin A or fragments thereof of at least 5 amino acids in a bodily fluid obtained from said subject; and
   correlating said level of Pro-Tachykinin A or fragments thereof with kidney function in a subject, or
   correlating said level of Pro-Tachykinin A or fragments thereof with kidney dysfunction wherein an elevated level above a certain threshold is predictive or diagnostic for kidney dysfunction in said subject, or
   correlating said level of Pro-Tachykinin A or fragments thereof with a risk of death or an adverse event in a diseased subject, wherein an elevated level above a certain threshold is predictive for an enhanced risk of death or adverse events, or
   correlating said level of Pro-Tachykinin A or fragments thereof with success of a therapy or intervention in a diseased subject, wherein a level below a certain threshold is predictive for a success of therapy or intervention, wherein said therapy or intervention may be renal replacement therapy or may be treatment with hyaluronic acid in patients having received renal replacement or predicting or monitoring the success of therapy or intervention may be prediction or monitoring recovery of renal function in patients with impaired renal function prior to and after renal replacement therapy and/or pharmaceutical interventions and/or adaption or withdrawal of nephrotoxic medications, or
   prediction of incidence of (chronic) kidney disease.
2. A method according to item 1, wherein said Pro-Tachykinin A is selected from the group comprising SEQ ID NO. 1 to 4 and fragments thereof are selected from the group comprising SEQ ID NO. 5 to 12.
3. A method according to items 1 to 2, wherein the level of Pro-Tachykinin A or fragments thereof of at least 5 amino acids is determined by using a binder to Pro-Tachykinin A or fragments thereof of at least 5 amino acids.
4. A method according to items 1 to 3, wherein the binder is selected from the group comprising an antibody, an antibody fragment or a non-Ig-Scaffold binding to Pro-Tachykinin A or fragments thereof of at least 5 amino acids.
5. A method according to any of items 1 to 4, wherein said binder binds to a region within the amino acid sequence selected from the group comprising SEQ ID NO. 5, SEQ ID NO. 11 and SEQ ID NO. 12.
6. A method according to any of the preceding items, wherein the threshold range is 80 to 100 pmol/L.
7. A method according to any of the preceding items, wherein the level of Pro-Tachykinin A is measured with an immunoassay and said binder is an antibody, or an antibody fragment binding to Pro-Tachykinin A or fragments thereof of at least 5 amino acids.
8. A method according to any of the items 1 to 7, wherein an assay is used comprising two binders that bind to two different regions within the region of Pro-Tachykinin A that is amino acid 3-22 (SEQ ID NO. 11) and amino acid 21-36 (SEQ ID NO. 12), wherein each of said regions comprises at least 4 or 5 amino acids.
9. A method according to any of items 1 to 8, wherein an assay is used for determining the level of Pro-Tachykinin A or fragments thereof of at least 5 amino acids and wherein the assay sensitivity of said assay is able to quantify the Pro-Tachykinin A or Pro-Tachykinin A fragments of healthy subjects and is <10 pmol/L.
10. A method according to any of items 1 to 9, wherein said bodily fluid may be selected from the group comprising blood, serum, plasma, urine, cerebrospinal fluid (CSF), and saliva.
11. A method according to items 1 to 10, wherein additionally at least one clinical parameter is determined selected from the group comprising: age, BUN, NGAL, PENK, creatinine clearance, creatinine and Apache Score.
12. A method according to any of items 1 to 11, wherein said determination is performed more than once in one patient.
13. A method according to any of items 1 to 12, wherein said monitoring is performed in order to evaluate the response of said subject to preventive and/or therapeutic measures taken.
14. A method according to any of items 1 to 13 in order to stratify said subjects into risk groups.
15. A point-of-care device for performing a method according to any of items 1 to 14, wherein said point of care device comprises at least two antibodies or antibody fragments directed to amino acid 3-22 (SEQ ID NO. 11) and amino acid 21-36 (SEQ ID NO. 12).
16. A kit for performing a method according to any of items 1 to 15, wherein said kit comprises at least two antibodies or antibody fragments directed to amino acid 3-22 (SEQ ID NO. 11) and amino acid 21-36 (SEQ ID NO.12).

### EXAMPLES

### Example 1

Development of Antibodies

Peptides

Peptides were synthesized (JPT Technologies, Berlin, Germany).

Peptides/Conjugates for Immunization

Peptides for immunization were synthesized (JPT Technologies, Berlin, Germany) with an additional N-terminal Cystein residue for conjugation of the peptides to bovine serum albumin (BSA). The peptides were covalently linked to BSA by using Sulfo-SMCC (Perbio-science, Bonn, Germany). The coupling procedure was performed according to the manual of Perbio.

TABLE 3

| Peptide for immunization | PTA Sequence |
|---|---|
| (C)GANDDLNYWSDWYDSDQIK | 3-22 (SEQ ID NO. 11) |
| (C) IKEELPEPFEHLLQRI | 21-36 (SEQ ID NO. 12) |

Generation of Monoclonal Antibodies

A BALB/c mouse was immunized with 100 μg peptide-BSA-conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intravenous injection.

Spenocytes from the immunized mouse and cells of the myeloma cell line SP2/0 were fused with 1 ml 50% polyethylene glycol for 30 s at 37° C. After washing, the cells were seeded in 96-well cell culture plates. Hybrid clones were selected by growing in HAT medium [RPMI 1640 culture medium supplemented with 20% fetal calf serum and HAT-supplement]. After two weeks the HAT medium is replaced with HT Medium for three passages followed by returning to the normal cell culture medium.

The cell culture supernatants were primary screened for antigen specific IgG antibodies three weeks after fusion. The positive tested microcultures were transferred into 24-well plates for propagation. After re-testing the selected cultures were cloned and recloned using the limiting-dilution technique and the isotypes were determined. (Lane, R. D. 1985: J. Immunol. Meth. 81: 223-228; Ziegler, B. et al. 1996: Horm. Metab. Res. 28: 11-15).

Antibodies were produced via standard antibody production methods (Marx et al., Monoclonal Antibody Production (1997), ATLA 25, 121) and purified via Protein A-chromatography. The antibody purities were >95% based on SDS gel electrophoresis analysis.

Labelling and Coating of Antibodies

All antibodies were labelled with acridinium ester according the following procedure:

Labelled compound (tracer, anti-PTA 3-22): 100 μg (100 μl) antibody (1 mg/ml in PBS, pH 7.4, was mixed with 10 μl Acridinium NHS-ester (1 mg/ml in acetonitrile, InVent GmbH, Germany) (EP 0353971) and incubated for 20 min at room temperature. Labelled antibody was purified by gel-filtration HPLC on Bio-Sil SEC 400-5 (Bio-Rad Laboratories, Inc., USA) The purified labelled antibody was diluted in (300 mmol/l potassiumphosphate, 100 mmol/l NaCl, 10 mmol/l Na-EDTA, 5 g/l bovine serum albumin, pH 7.0). The final concentration was approx. 800.000 relative light units (RLU) of labelled compound (approx. 20 ng labeled antibody) per 200 μl. Acridiniumester chemiluminescence was measured by using an AutoLumat LB 953 (Berthold Technologies GmbH & Co. KG).

Solid Phase Antibody (Coated Antibody)

Solid phase: Polystyrene tubes (Greiner Bio-One International AG, Austria) were coated (18 h at room temperature) with anti PTA 22-36 antibody (1.5 μg antibody/0.3 ml 100 mmol/1 NaCl, 50 mmol/l Tris/HCl, pH 7.8). After blocking with 5% bovine serum albumine, the tubes were washed with PBS, pH 7.4 and vacuum dried.

Pro-Tachykinin A Immunoassay

50 μl of sample (or calibrator) was pipetted into coated tubes, after adding labeled antibody (200 ul), the tubes were incubated for 2 h at 18-25° C. Unbound tracer was removed by washing 5 times (each 1 ml) with washing solution (20 mmol/l PBS, pH 7.4, 0.1% Triton X-100). Tube-bound labelled antibody was measured by using a Luminometer LB 953, Berthold, Germany.

Calibration:

The assay was calibrated, using dilutions of synthetic P37, diluted in 20 mM K₂PO₄, 6 mM EDTA, 0.5% BSA, 50 μM Amastatin, 100 μM Leupeptin, pH 8.0. PTA control plasma is available at ICI-diagnostics, Berlin, Germany.

FIG. 1 shows a typical PTA dose/signal curve.

The analytical assay sensitivity was (the median signal generated by 20 determinations of 0-calibrator (no addition of PTA)+2SD2 standard deviations (SD), the corresponding PTA concentration is calculated from a standard curve) 4.4 pmol/L.

Creatinine Clearance

Creatinine clearance was determined using the MDRD formula (see Levey et al. 2009. Ann Intern Med. 150(9): 604-612).

### Example 2

PTA in Healthy Subjects

EDTA-plasma samples from fasting healthy subjects (n=4435, average age 56 years) were measured using the PTA assay. The mean value of PTA in the population was 55.2 pmol/L, standard deviation+/−17.8 pmol/L, the lowest value was 9.07 pmol/L and the 99^{th} percentile was 107.6 pmol/L. All values were detectable with the assay, since the assay sensitivity was 4.4 pmol/L. The distribution of PTA values in healthy subjects is shown in FIG. 2.

Surprisingly, Pro-Tachykinin A was negatively correlated with eGFR in healthy subjects (r=−0.23, p<0.0001), see FIG. 3. The coefficient of correlation was comparable in male and females (r=0.22 vs 0.21, both p<0.0001). These data indicating a strong association between PTA and kidney function.

### Example 3

Correlation of PTA and Kidney Function (Creatinine Clearance) in Patients with Chronic and Acute Diseases.

TABLE 4

| Disease | r-value | p-value |
|---|---|---|
| Acute Myocardial Infarction | −0.56 | <0.0001 |
| Sepsis | −0.58 | <0.0001 |
| N = 101 | | |
| Chronic heart failure | −0.43 | <0.0001 |

PTA correlated always significantly with creatinine clearance, in acute diseases the correlation was stronger than in chronic diseases or in healthy subjects.

### Example 4

PTA in Sepsis Patients

To investigate the diagnostic performance of PTA for diagnosis of kidney failure in acute clinical settings, we performed the following clinical study:

101 ED patients fulfilling the definition of sepsis (Dellinger et al. 2008. Crit Care Med 3(1):296-327) were subsequently hospitalized (average 5 days of hospitalization) and received a standard of care treatment. EDTA-plasma was generated from day 1 (ED presentation) and one sample each day during hospital stay. The time to freeze samples for later analyte-measurement was less than 4 h.

Patient characteristics are summarized in Table 5:

TABLE 5
Patients characteristics of sepsis patients

| | | in hospital | | |
|---|---|---|---|---|
| | all | deaths | discharged | p- |
| Variable | (n = 101) | (n = 27) | (n = 74) | value |
| Demographics | | | | |
| Gender - male | 60 (60) | 13 (48) | 47 (64) | 0.163 |
| Age - median [IQR] | 78 [72-72] | 77 [71.25-83] | 80 [75-84.5] | 0.142 |
| Examination variables | | | | |
| BP systolic (mmHg) - | 115 [100-100] | 120 [106.25-138.75] | 105 [80-120] | 0.001 |
| median [IQR] | | | | |
| BP diastolic (mmHg) - | 65 [60-60] | 65 [60-85] | 60 [50-70] | 0.002 |
| median [IQR] | | | | |
| HR - median [IQR] | 100 [94-94] | 100 [94-114.75] | 100 [93.5-107.5] | 0.407 |
| RR - median [IQR] | 24 [22-22] | 24 [22-28] | 26 [24-28] | 0.069 |
| MAP (mmHg) - median [IQR] | 83.3 [74-74] | 83.3 [77.62-100.75] | 81.6 [63.5-89] | 0.026 |
| concomitant diseases | | | | |
| Cardiovascular - yes | 26 (25.7) | 9 (33.3) | 17 (23) | 0.311 |
| Hypertensive - yes | 47 (46.5) | 13 (48.1) | 34 (45.9) | 1.000 |
| Diabetes - yes | 35 (34.7) | 9 (33.3) | 26 (35.1) | 1.000 |
| Cancere - yes | 13 (12.9) | 3 (11.1) | 10 (13.5) | 1.000 |
| routine labaratory variables | | | | |
| Blood culture - yes | 31 (31) | 5 (19) | 26 (35) | 0.246 |
| negative | 15 (16.3) | 2 (8) | 13 (19.4) | |
| positive | 16 (17.4) | 3 (12) | 13 (19.4) | |
| Creatinine clearance (ml/min) - | 48 [23.25-23.25] | 56 [29.25-80] | 31.5 [14.75-66] | 0.043 |
| median [IQR] | | | | |
| Creatinine - median [IQR] | 1.3 [0.9-0.9] | 1.25 [0.9-2.08] | 1.8 [1-3.15] | 0.080 |
| UREA - median [IQR] | 36 [21-21] | 31.5 [20-53.25] | 51 [42-87] | 0.004 |
| GCS - median [IQR] | 15 [10-10] | 15 [12.5-15] | 8 [8-11] | <0.001 |
| Pcr - median [IQR] | 16 [6.6-6.6] | 14.5 [6.7-23.7] | 17.35 [6.6-28.05] | 0.846 |
| Glucose - median [IQR] | 113.5 [94.5-94.5] | 110 [95.5-144] | 128 [94-160.5] | 0.400 |
| bilirubin - median [IQR] | 0.9 [0.71-0.71] | 0.9 [0.7-1.03] | 0.91 [0.77-1.18] | 0.534 |
| GR - median [IQR] | 3.8 [3.3-3.3] | 3.8 [3.2-4.3] | 3.7 [3.4-4.2] | 0.684 |
| GB - median [IQR] | 12700 [6774-6774] | 13100 [8115-17565] | 11920 [25.55-18790] | 0.343 |
| PLT - median [IQR] | 213 [150-150] | 217 [154.75-301] | 185 [130-236.5] | 0.113 |
| HCT - median [IQR] | 32 [28-28] | 31.5 [28-37] | 34 [31.25-39.5] | 0.149 |
| Leuco/Neutr (%) - median [IQR] | 87 [80-80] | 86 [78.25-89.95] | 91 [87-93.05] | 0.001 |
| HB - median [IQR] | 10.4 [9.47-9.47] | 10.15 [9.3-12.4] | 10.85 [9.9-12.67] | 0.220 |
| Na - median [IQR] | 137 [134-134] | 137 [133-141] | 139 [134-144.5] | 0.204 |
| K - median [IQR] | 3.9 [3.5-3.5] | 3.9 [3.6-4.3] | 3.9 [3.3-5.1] | 0.982 |
| INR - median [IQR] | 1.19 [1.1-1.1] | 1.19 [1.1-1.4] | 1.18 [1.04-1.36] | 0.731 |
| TC - median [IQR] | 38.4 [36-36] | 38.5 [38.12-38.7] | 36 [35.55-38.5] | <0.001 |
| SAO₂ - median [IQR] | 94 [90-90] | 95 [90.25-97] | 93 [88.5-95.5] | 0.119 |
| pH - median [IQR] | 7.45 [7.38-7.38] | 7.46 [7.4-7.5] | 7.4 [7.24-7.4] | <0.001 |
| PO₂ - median [IQR] | 67 [56-56] | 66.5 [56-78] | 67 [56.5-79.5] | 0.806 |
| PCO₂ -median [IQR] | 36 [32-32] | 37.5 [33-43.75] | 34 [30-41] | 0.245 |
| Lactate - median [IQR] | 1.5 [1-1] | 1.3 [0.83-1.9] | 2.5 [1.4-4.15] | <0.001 |
| Bic - median [IQR] | 23.5 [21-21] | 24.25 [21.43-28] | 21 [17.35-23.25] | 0.001 |
| FiO₂ (%) - median [IQR] | 21 [21-21] | 21 [21-23.25] | 24 [21-45] | <0.001 |
| other | | | | |
| Acute organ dysfunction - yes | 39 (43.3) | 16 (64) | 23 (35.4) | 0.021 |
| Apache score (%) - median [IQR] | 19 [12.5-12.5] | 14.65 [12.12-20.38] | 32 [20-39] | <0.001 |
| Days hospitalized - median [IQR] | 5 [2-2] | 6 [4-7] | 2 [1-6] | 0.003 |
| treatment at baseline | | | | |
| Diuresis (cc) - median [IQR] | 900 [600-600] | 1000 [700-1200] | 450 [200-1025] | <0.001 |
| Steroids-yes | 16 (15.8) | 4 (14.8) | 12 (16.2) | 1.000 |
| Vasopressors - yes | 18 (17.8) | 13 (48.1) | 5 (6.8) | <0.001 |
| Antibiotics - yes | 101 (100) | 27 (100) | 74 (100) | 1.000 |
| Fluid therapy - yes | 101 (100) | 27 (100) | 74 (100) | 1.000 |

26.7% of all patients died during hospital stay and are counted as treatment non-responder,

73.3% of all patients survived the sepsis and are counted as treatment responder.

50% of all patients presenting with sepsis had a PTA value>107 pmol/L (99 percentile), indicating PTA not to be a marker for the infection.

Results of Clinical Study

PTA highly correlated to creatinine clearance (r=−0.58, p<0.0001, FIG. 4).

Kidney dysfunction was defined based on the RIFLE criteria (Venkataraman and Kellum, 2007 J Intensive Care Med. 22(41:187-93). Patients were counted as kidney dysfunction if any of the RIFLE classification factors was fulfilled. Within the study cohort, we determined the RIFLE within 90 subjects at day 1 (presentation at ED), 39 patients fulfilled RIFLE classification (had risk of kidney disease, kidney injury, kidney failure loss of kidney function or end-stage kidney disease) and 51 patients had no kidney dysfunction. Increased PTA was significantly (p=<0.0001) correlated with kidney dysfunction (AUC: 0.787) (FIG. 5).

### Example 5

PTA in Patients Admitted to the Emergency Department (ED)

This was a prospective, observational trial enrolling 97 patients consecutively admitted to the emergency department of Sant' Andrea Hospital in Rome for acute pathological conditions, and further hospitalization. For each enrolled patient clinical laboratory data and plasma PTA values were collected at arrival. The patient's characteristics are summarized in Table 6. A phone call 60-day follow-up was performed after hospital discharge.

TABLE 6
Patient's characteristics (ED trial)

| Variable | N = 97 |
|---|---|
| Age (years) | 76 +/−12 |
| Gender (male) | 60% |
| 60 day survival rate | 81.4% |
| Final diagnosis | |
| AHF | 40.2% |
| Sepsis | 21.6% |
| Local infection | 18.6% |
| Gastrointestinal disorders | 10.3% |
| other | 9.3% |
| Renal SOFA Score | |
| 0 | 43.3% |
| 1 | 30.9% |
| 2 | 15.5% |
| >2 | 10.3% |

The survival rate was 81.4% and events (death) occurred mainly in the first week after admission to the hospital. PTA was measured on admission. PTA values correlated with the severity/stage of acute kidney injury according to the RIFLE criteria (FIG. 6 a) and AKIN classification (FIG. 6 b).

We correlated the initial PTA value with the in hospital mortality. PTA is highly prognostic for outcome in hospitalized ED patients (see FIG. 7) (AUC/C index 0.795; p<0.00001). PTA is substantially stronger in prognosis than the NGAL and even stronger than PENK (see Table 7).

TABLE 7

| Model | Model Chi² | p-value | C-index [95% CI] |
|---|---|---|---|
| age | 0 | 0.95 | 0.59 [0.43-0.75] |
| NGAL (pg/mL) | 18.1 | 0.00002 | 0.78 [0.69-0.90] |
| eGFR | 20.5 | 0.00001 | 0.75 [0.61-0.88] |
| PENK (pmol/L) | 23.4 | <0.00001 | 0.79 [0.69-0.89] |
| PTA (pmol/L) | 27.4 | <0.00001 | 0.80 [0.67-0.92] |
| APACHE II Score | 30.4 | <0.00001 | 0.82 [0.71-0.92] |

FIG. 8 shows a Kaplan-Meier-Plot for survival of ED patients according to a) quartiles of PTA on admission and b) Cut-off of 100 pmol/L of PTA on admission.

There is a significant added information if PTA and PENK are combined (p=0.004) and if PTA and APACHE II-Score are combined (p=0.001).

We correlated the initial PTA value with the RIFLE criteria. PTA is highly diagnostic for acute kidney injury in hospitalized ED patients (AUC/C-index 0.792; p<0.00001) and substantially stronger (p<0.0001) than the marker PENK that revealed an AUC/C-index of 0.66 (p=0.002).

### Example 6

Diagnosis and Prognosis of CKD

Study Population

The background population for this study is the population-based prospective study from Malmö, Sweden, (Malmö Diet and Cancer Study MDCS) of which 28,098 healthy men and women born between 1923-1945 and 1923-1950 participated in the baseline examination between 1991 and 1996. The total participation rate was approximately 40.8%. Individuals from 6,103 randomly selected participants of the MDCS who underwent additional phenotyping were included, designed to study epidemiology of carotid artery disease, in the MDC Cardiovascular Cohort (MDC-CC) between 1991 and 1994. During the follow-up re-examination this random sample was re-invited to the follow-up re-examination between 2007 and 2012. 3,734 individuals of those that were alive and had not emigrated from Sweden (N=4,924) attended the follow-up re-examination. After excluding all individuals without PTA levels measured at baseline (n=1,664), the association between yearly change in eGFR, plasma creatinine and plasma Cystatin C in 2,492 individuals was tested, for whom measurements where available at both examinations. The relation between PTA concentration at baseline and incident CKD at follow-up re-examination was examined in a total of 2,459 participants with an eGFR of higher than 60 ml/min/1.73 m² at baseline.

All participants underwent a physical examination during baseline examination and the following anthropometric characteristics were assessed: height (cm), weight (kg), waist as well as hip circumference by trained nurses. Systolic and diastolic blood pressure (mmHG) were measured after 10 minutes of rest by trained personal. Lean body mass and body fat were estimated using a bioelectric impedance analysis (single-frequence analyses, BIA 103; JRL Systems, Detroit, Mich.). Questions concerning socio-economic status, lifestyle factors and medical history were answered by the participants via self-administrated questionnaire. Non-fasting-blood samples were drawn and immediately frozen to −80° C. and stored in a biological bank available for DNA extraction. Participant in the MDC-CC also provided fasting blood samples in which plasma creatinine (pmol/L) and cystatin C (mg/L) were measured. In addition total cholesterol (mmol/L), Triglyceride (TG) (mmol/L), low-density-lipo-cholesterol (LDL-C) (mmol/L), high-density-lipo-cholesterol (HDL-C) (mmol/L), whole blood glucose (mmol/L), plasma insulin (μlU/ml), HOMA (insulin*glucose/22.5), HbA1c (%) were quantified and blood pressure was measured in supine position with a mercury column sphygmomanometer after 10 min of rest.

During the follow-up re-examination (2007-2012) the following anthropometric characteristics were measured: height (m), weight (kg), waist and hip circumference (cm), systolic and diastolic blood pressure (SBP and DBP) (mmHG) following a similar protocol as in the baseline examination. Further concentrations of cholesterol (mmol/L), triglyceride (mmol/L), HDL-C (mmol/L), glucose (mmol/L), Creatinine (mmol/L), Cystatin C (mg/1) were quantified in fasting blood samples.

PTA was measured in fasting plasma samples from 4,446 participants at MDC-CC baseline examination using the chemiluminometric sandwich immunoassay. For 1,664 individuals fasting plasma levels of PTA were lacking. Those were slightly younger, had a marginal higher BMI and plasma creatinine as well as lower systolic blood pressure, fasting glucose and HbA1c-concentration at MDC baseline but did not differ in gender, plasma lipids, cystatin C or anti-hypertensive treatment frequency levels from the included participants. To achieve normal distribution we transformed the positively skewed concentration of fasting plasma PTA with the logarithm, base 10. Additionally, continuous PTA concentrations were divided into tertiles, defining the first tertile (lowest PTA concentration) as the reference. Both, at baseline and follow-up examination, concentrations of creatinine and cystain C were analyzed from plasma and are presented in μmol/L and mg/L, respectively. CKD was defined as presence of an estimated GFR (eGFR) of less than 60 ml/min/1.73 m² calculated according to the previously reported CKD-EPI-2012 equation which considers blood concentration of creatinine as well as cystatin C.

Statistical Analyses

Association between fasting plasma PTA concentration at baseline and the risk of CKD at follow-up re-examination was analyzed using logistic regression adjusting for follow-up time in years, age, sex, GFR (ml/min/1.73 m²) and for common risk factors for kidney function at baseline (systolic blood pressure, BMI (kg/m2), fasting glucose and anti-hypertensive medication).

Equation: Example Mean Change in Weight (Kg) Per Year of Follow-Up

$$\frac{{weight ( kg )}_{follow \text{-} up re \text{-} examination} - {weight ( kg )}_{baseline examination}}{follow \text{-} up time ( years )}$$

SPSS (version 21, IBM) was used for the clinical epidemiological analyses and all analyses were adjusted for sex and age. Additional adjustments for covariates in specific models are reported in the results section. The null-hypothesis was rejected, if a 2-sided P-value of less than 0.05 was observed and the association was considered as statistical significant.

Cross-Sectional Analyses Between PTA and Kidney Function at MDC Baseline (1991-1994)

High levels of PTA were significantly associated with older age and decrease in several anthropometric characteristics. In addition, concentrations of TG, fasting plasma glucose, plasma insulin and HBbA1c decreased with increasing PTA. Creatinine and cystatin C levels were significantly higher for individuals in the highest tertile (Table 8).

TABLE 8
Cross-sectional relationship between tertiles of PTA levels and
phenotypic characteristics of Malmö Diet and Cancer Study
participants baseline¹ (1991-1994)

| | | Fasting plasma PTA concentration | | | |
|---|---|---|---|---|---|
| | n | Low | Medium | High | P |
| Age (years) | 4634 | 56.96 (6.02) | 57.91 (5.93) | 58.56 (5.87) | <0.0001 |
| BMI (kg/m²) | 4630 | 26.52 (4.21) | 25.84 (3.78) | 25.21 (3.7) | <0.0001 |
| SBP (mmHG) | 4634 | 143.08 (18.99) | 141.8 (19.24) | 142.31 (19.56) | 0.189 |
| DBP (mmHG) | 4634 | 88.15 (9.71) | 86.85 (9.35) | 86.48 (9.48) | <0.0001 |
| Glucose | 4616 | 5.39 (1.57) | 5.2 (1.41) | 5.06 (1.16) | <0.0001 |
| (mmol/L)⁴ | | | | | |
| Creatinine | 4541 | 83.74 (14.6) | 83.02 (14.02) | 85.85 (19.59) | <0.0001 |
| (μmol/L) | | | | | |
| Cystatin C (mg/L) | 4310 | 0.75 (0.12) | 0.78 (0.13) | 0.83 (0.19) | <0.0001 |
| eGFR CKD-EPI | 4252 | 92.66 (12.95) | 89.47 (12.79) | 84.61 (13.66) | <0.0001 |
| 2012 | | | | | |
| Antihypertensive | 760 (17.1) | 285 (19.2) | 236 (15.9) | 239 (16.2) | 0.029² |
| treatment (%) | | | | | |

¹as mean and SD; ⁴fasting whole blood was converted into plasma value by multiplication with the factor 1.11; SBP = Systolic blood pressure; DBP = Diastolic blood pressure; ²Chi²-test

Prospective changes in kidney function at follow-up re-examination in relation to fasting plasma PTA concentration at baseline examination

Next, the relation between fasting plasma PTA concentration at baseline and change for phenotypic characteristics between baseline and follow-up re-examination in 2,908 participants from MDC-CC was examined (Table 9).

TABLE 9
Association between tertiles of fasting plasma PTA
at baseline examination and mean changes by year in kidney
function and other clinical characteristics during the follow
up re-examination in Malmo Diet and Cancer Study

| | | Fasting plasma PTA concentration | | | |
|---|---|---|---|---|---|
| | n | Low | Medium | High | P |
| N (%) | | 971 (33.4) | 965 (33.2) | 972 (33.2) | |
| BMI (kg/m²) | 2455 | −0.1 (0.2) | −0.1 (0.2) | −0.1 (0.2) | 0.253 |
| SBP (mmHG) | 2454 | −0.19 (1.33) | −0.28 (1.26) | −0.19 (1.28) | 0.254 |
| DBP (mmHG) | 2453 | 0.22 (0.74) | 0.16 (0.71) | 0.21 (0.72) | 0.266 |
| Glucose (mmol/L)² | 2189 | −0.15 (0.16) | −0.14 (0.14) | −0.14 (0.14) | 0.259 |
| Creatinine (μmol/L) | 2459 | 0.07 (1.12) | 0.02 (1.06) | −0.04 (1.76) | 0.274 |
| Cystatin C (mg/L) | 2459 | −0.02 (0.01) | −0.02 (0.01) | −0.02 (0.02) | 0.616 |
| eGFR CKD-EPI 2012 | 2459 | 1.52 (0.85) | 1.47 (0.8) | 1.41 (0.83) | 0.019 |
| Incidence of CKD (%) | 788 | 228 (27.8) | 250 (30.6) | 310 (37.7) | 0.0001 |
| | (32) | | | | |

BSA = body surface area; ²Difference was calculated transferring the baseline fasting whole blood into plasma value (x factor 1.11); SBP = Systolic blood pressure; DBP = Diastolic blood pressure

Prospective Analysis of the Association Between Fasting Plasma PTA Levels at Baseline and CKD at Follow-Up Re-Examination

Prevalence of CKD based on eGFR above 60 ml/min/1.73 m² was 32.0% (n=788) in 2,459 participants during a median follow-up time of 16.5 years (range 13.3-20.2 years). We observed a significant risk increase for incidence of CKD at follow-up re-examination with increasing PTA levels in a logistic regression model (standardized OR (per increase in 1 IQR): 1.22, 95% CI 1.1-1.4; P=0.0005, AUC=0.554).

In a cohort of MDC Study (n=4340) PTA was measured at baseline and correlated to the diagnosis of CKD. PTA values were significantly associated with CKD-stage (estimated GFR), with highest values in patients with eGFR ranging between 15 and 30 (FIG. 9).

### Example 7

Val-HeFT-Study

Val-HeFT was a randomized, placebo-controlled, double-blinded, multicenter trial that enrolled 5010 patients with symptomatic HF to evaluate the efficacy of the ARB valsartan. Briefly, patients over the age of 18 years, in stable NYHA class II-IV HF, LVEF 40%, and LV internal diastolic dimension (LVIDD)/body surface area (BSA) 2.9 cm/m2 on echocardiography were eligible. All patients had to be receiving stable pharmacological treatment for HF. The Val-HeFT had two primary endpoints: all-cause mortality and the first morbid event, which was defined as death, sudden death with resuscitation, hospitalization for HF, or administration of an i.v. inotropic or vasodilator drug for ≥4 h without hospitalization. Hospitalization for HF was a secondary endpoint (Cohn and Tognoni 2001. N Engl J Med 345:1667-1675). In the Val-HeFT, valsartan had no effect on mortality, but reduced the first morbid events by 13% and hospitalizations for HF by 28%.

In patients with chronic heart failure there was a strong correlation with creatinine (r=0.41, p<0.0001) and eGFR (r=−0.43, p<0.0001).

### Example 8

ADRENOSS Study (Adrenomedullin and Outcome in Severe Sepsis and Septic Shock)

596 patients admitted in intensive care unit of 26 hospitals, in 5 countries, with diagnosis of severe sepsis or septic shock, were included in this study. Inclusion Criteria were: age>18 years, patients admitted in intensive care unit for severe sepsis or septic shock according to international, standardized criteria, transferred from another intensive care unit less than 24 hours after the primary admission, or being treated with vasopressors for less than 24 hours in the prior ICU, signed consent form. Exclusion criteria were: age<18 years, severe sepsis or septic shock patients transferred from another intensive care unit later than 24 hours after the primary admission or being treated with vasopressors for more than 24 hours in the prior ICU, pregnant women, vegetative coma, participation in an interventional clinical trial in the preceding month.

Primary outcome measure was the rate of all-cause mortality (time-frame day 28). Plasma samples (heparin-, EDTA-, EDTA/aprotinin plasma) and urine samples were collected on admission, day 2, day 3 and the day of discharge for measuring biomarkers.

EDTA-plasma samples from 577 patients were available on admission. Median concentration of PTA in this cohort was 115.5 pmol/L. PTA values were significantly correlated to creatinine levels (r=0.56; p<0.0001).

By definition, worsening of renal function (WRF) occurred when the serum creatinine level increased during hospitalization by 0.3 mg/dL and by > or =25% from admission.

PTA predicted worsening of renal function (AUC 0.603) using a PTA cut-off at 100 pmol/L and was significantly better than creatinine alone. Adding PTA to creatinine added significant value (p<0.05).

### FIGURE DESCRIPTION

FIG. 1: A typical Pro-Tachykinin A dose/signal curve.

FIG. 2: Frequency distribution of Pro-Tachykinin A in a healthy population (n=4463)

FIG. 3: Correlation of eGFR and PTA in healthy subjects. x-axis: quartiles of eGFR, y-axis: quartiles of PTA

FIG. 4: PTA highly correlated to creatinine clearance in the sepsis cohort (r=−0.58, p<0.0001).

FIG. 5: PTA for diagnosis of kidney dysfunction in sepsis

FIG. 6 a): Correlation of PTA levels with RIFLE criteria (ED trial)

FIG. 6 b): Correlation of PTA levels with AKIN criteria (ED trial)

FIG. 7: PTA for prognosis of mortality in ED patients

FIG. 8 a): Kaplan-Meier-Plot for survival of ED patients on admission (according to PTA quartiles)

FIG. 8 b): Kaplan-Meier-Plot for survival of ED patients on admission (PTA Cut-off 100 pmol/L)

FIG. 9: Diagnosis of CKD

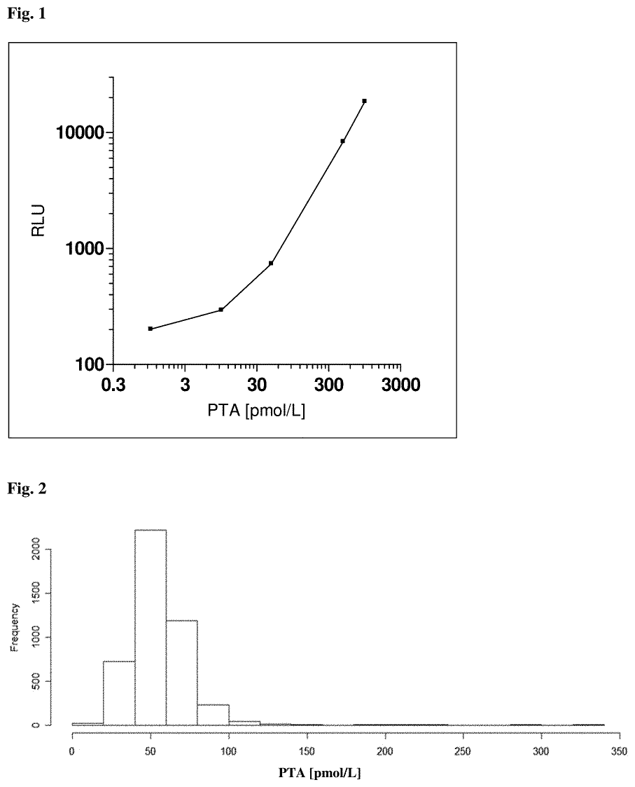

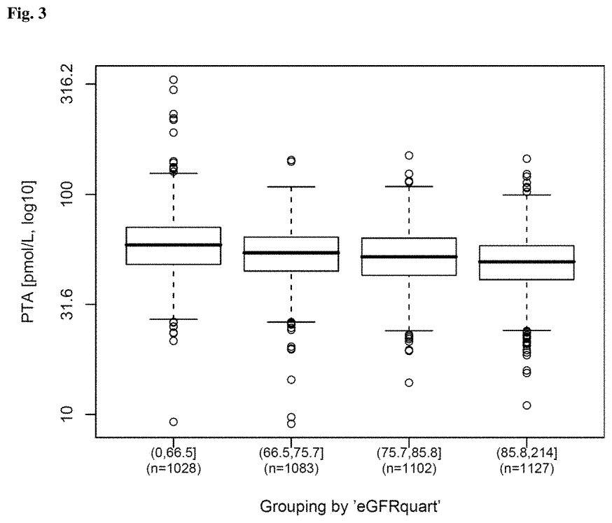

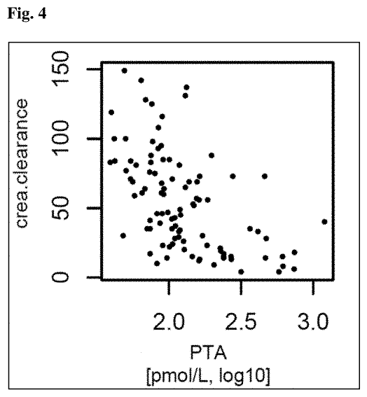

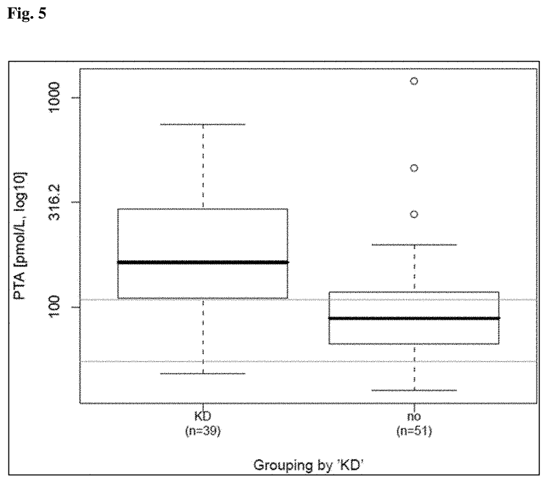

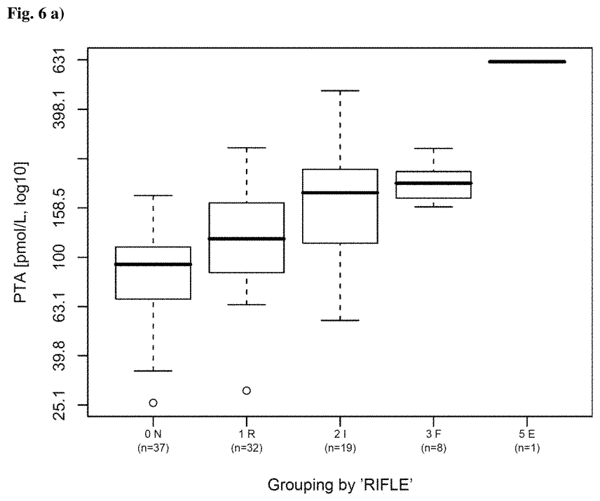

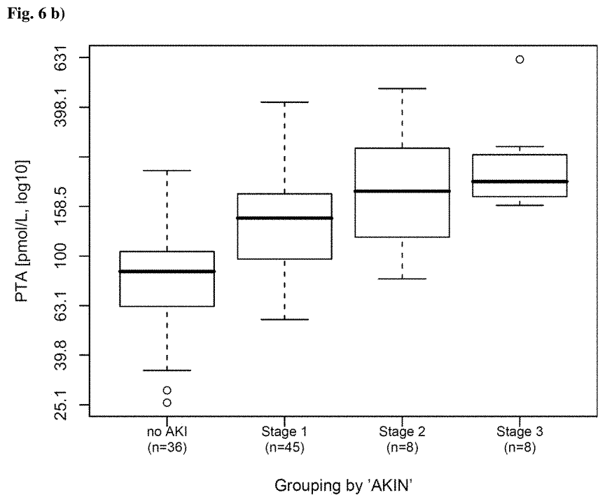

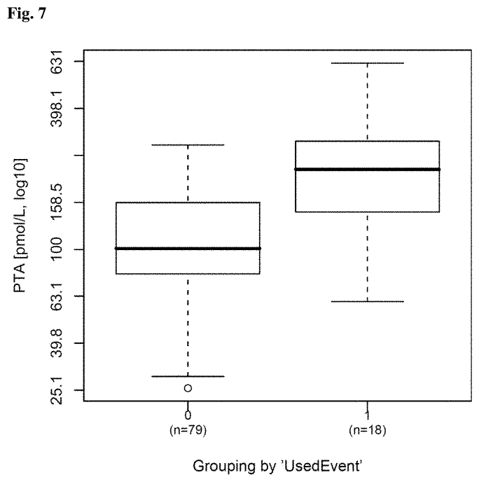

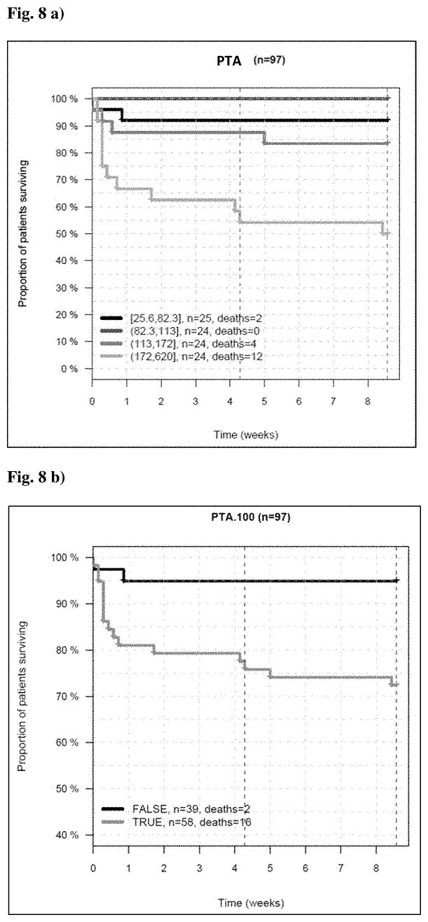

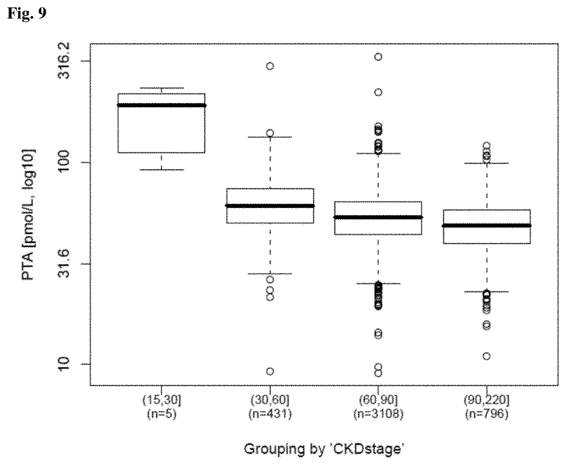

1. A method for (a) diagnosing or monitoring kidney function in a subject or (b) diagnosing kidney dysfunction in a subject or (c) predicting the risk of death or an adverse event in a diseased subject, wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or (d) predicting or monitoring the success of a therapy or intervention or (e) predicting incidence of (chronic) kidney disease comprising
determining the level of Pro-Tachykinin A or fragments thereof of at least 5 amino acids in a bodily fluid obtained from said subject; and
diagnosing or monitoring kidney function in said subject based on the level of Pro-Tachykinin A or fragments thereof in the bodily fluid obtained from said subject, or
diagnosing kidney dysfunction in said subject based on the level of Pro-Tachykinin A or fragments thereof in the bodily fluid obtained from said subject wherein an elevated level above a certain threshold is predictive or diagnostic for kidney dysfunction in said subject, or
predicting the risk of death or an adverse event in a subject based on the level of Pro-Tachykinin A or fragments thereof in the bodily fluid obtained from said subject, wherein an elevated level above a certain threshold is predictive for an enhanced risk of death or adverse events and wherein said subject is a diseased subject and wherein said adverse event is selected from the group comprising worsening of kidney dysfunction including kidney failure, loss of kidney function and end-stage kidney disease or death due to kidney dysfunction including kidney failure, loss of kidney function and end-stage kidney disease, or
predicting or monitoring the success of a therapy or intervention based on the level of Pro-Tachykinin A or fragments thereof in the bodily fluid obtained from said subject, wherein a level below a certain threshold is predictive for a success of therapy or intervention, wherein said subject is a diseased subject and wherein said therapy or intervention is selected from the group comprising renal replacement therapy, and treatment with hyaluronic acid in patients having received renal replacement therapy, or
predicting or monitoring the recovery of renal function in subjects with impaired renal function prior to and after renal replacement therapy, pharmaceutical interventions and/or adaption of withdrawal of nephrotoxic medications, based on the level of Pro-Tachykinin A or fragments thereof in the bodily fluid obtained from said subject, or
predicting the incidence of chronic kidney disease based on the level of Pro-Tachykinin A or fragments thereof in the bodily fluid obtained from said subject.

2. A method according to claim 1, wherein said Pro-Tachykinin A is selected from the group comprising SEQ ID NO. 1 to 4 and fragments thereof are selected from the group comprising SEQ ID NO. 5 to 12.

3. A method according to claim 1, wherein the level of Pro-Tachykinin A or fragments thereof of at least 5 amino acids is determined by using a binder to Pro-Tachykinin A or fragments thereof of at least 5 amino acids.

4. A method according to claim 1, wherein the binder is selected from the group comprising an antibody, an antibody fragment or a non-Ig-Scaffold binding to Pro-Tachykinin A or fragments thereof of at least 5 amino acids.

5. A method according to claim 1, wherein said binder binds to a region within the amino acid sequence selected from the group comprising SEQ ID NO. 5, SEQ ID NO. 11 and SEQ ID NO. 12.

6. A method according to claim 1, wherein the threshold range is 80 to 100 pmol/L.

7. A method according to claim 1, wherein the level of Pro-Tachykinin A is measured with an immunoassay and said binder is an antibody, or an antibody fragment binding to Pro-Tachykinin A or fragments thereof of at least 5 amino acids.

8. A method according to claim 1, wherein an assay is used comprising two binders that bind to two different regions within the region of Pro-Tachykinin A that is amino acid 3-22 (SEQ ID NO. 11) and amino acid 21-36 (SEQ ID NO. 12), wherein each of said regions comprises at least 4 or 5 amino acids.

9. A method according to claim 1, wherein an assay is used for determining the level of Pro-Tachykinin A or fragments thereof of at least 5 amino acids and wherein the assay sensitivity of said assay is able to quantify the Pro-Tachykinin A or Pro-Tachykinin A fragments of healthy subjects and is <10 pmol/L.

10. A method according to claim 1, wherein said bodily fluid may be selected from the group comprising blood, serum, plasma, urine, cerebrospinal fluid (CSF), and saliva.

11. A method according to claim 1, wherein additionally at least one clinical parameter is determined selected from the group comprising: age, blood urea nitrogen (BUN), neutrophil gelatinase-associated lipocalin (NGAL), proenkephalin (PENK), creatinine clearance, creatinine and Apache Score.

12. A method according to claim 1, wherein said determination is performed more than once in one patient.

13. A method according to claim 1, wherein said monitoring is performed in order to evaluate the response of said subject to preventive and/or therapeutic measures taken.

14. A method according to claim 1 in order to stratify said subjects into risk groups.

15. A point-of-care device for performing a method according to claim 1, wherein said point of care device comprises at least two antibodies or antibody fragments directed to amino acid 3-22 (SEQ ID NO. 11) and amino acid 21-36 (SEQ ID NO. 12).

16. A kit for performing a method according to claim 1, wherein said kit comprises at least two antibodies or antibody fragments directed to amino acid 3-22 (SEQ ID NO. 11) and amino acid 21-36 (SEQ ID NO.12).


SEQUENCE LISTING (24 sequences)

SEQ ID NO: 1 - protein, 92 aa, Homo sapiens
EEIGANDDLNYWSDWYDSDQIKEELPEPFEHLLQRIARRPKPQQFFGLMGKRDADSSIEKQVALLKALYGHGQISHKMAYERSAMQNYERRR

SEQ ID NO: 2 - protein, 110 aa, Homo sapiens
EEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 3 - protein, 98 aa, Homo sapiens
EEIGANDDLNYWSDWYDSDQIKEELPEPFEHLLQRIARRPKPQQFFGLMGKRDAGHGQISHKRHKTDSFVGLMGKRALNSVAYERSAMQNYERRRSEQ

SEQ ID NO: 4 - protein, 77 aa, Homo sapiens
EEIGANDDLNYWSDWYDSDQIKEELPEPFEHLLQRIARRPKPQQFFGLMGKRDAGHGQISHKMAYERSAMQNYERRR

SEQ ID NO: 5 - protein, 37 aa, Homo sapiens
EEIGANDDLNYWSDWYDSDQIKEELPEPFEHLLQRIA

SEQ ID NO: 6 - protein, 11 aa, Homo sapiens
RPKPQQFFGLM

SEQ ID NO: 7 - protein, 36 aa, Homo sapiens
DADSSIEKQVALLKALYGHGQISHKRHKTDSFVGLM

SEQ ID NO: 8 - protein, 19 aa, Homo sapiens
GHGQISHKRHKTDSFVGLM

SEQ ID NO: 9 - protein, 10 aa, Homo sapiens
HKTDSFVGLM

SEQ ID NO: 10 - protein, 16 aa, Homo sapiens
ALNSVAYERSAMQNYE

SEQ ID NO: 11 - protein, 19 aa, Homo sapiens
GANDDLNYWSDWYDSDQIK

SEQ ID NO: 12 - protein, 16 aa, Homo sapiens
IKEELPEPFEHLLQRI

SEQ ID NO: 13 - protein, 243 aa, Homo sapiens
ECSQDCATCSYRLVRPADINFLACVMECEGKLPSLKIWETCKELLQLSKPELPQDGTSTLRENSKPEESHLLAKRYGGFMKRYGGFMKKMDELYPMEPEEEANGSEILAKRYGGFMKKDAEEDDSLANSSDLLKELLETGDNRERSHHQDGSDNEEEVSKRYGGFMRGLKRSPQLEDEAKELQKRYGGFMRRVGRPEWWMDYQKRYGGFLKRFAEALPSDEEGESYSKEVPEMEKRYGGFMRF

SEQ ID NO: 14 - protein, 73 aa, Homo sapiens
ECSQDCATCSYRLVRPADINFLACVMECEGKLPSLKIWETCKELLQLSKPELPQDGTSTLRENSKPEESHLLA

SEQ ID NO: 15 - protein, 5 aa, Homo sapiens
YGGFM

SEQ ID NO: 16 - protein, 5 aa, Homo sapiens
YGGFL

SEQ ID NO: 17 - protein, 20 aa, Homo sapiens
MDELYPMEPEEEANGSEILA

SEQ ID NO: 18 - protein, 41 aa, Homo sapiens
DAEEDDSLANSSDLLKELLETGDNRERSHHQDGSDNEEEVS

SEQ ID NO: 19 - protein, 8 aa, Homo sapiens
YGGFMRGL

SEQ ID NO: 20 - protein, 12 aa, Homo sapiens
SPQLEDEAKELQ

SEQ ID NO: 21 - protein, 11 aa, Homo sapiens
VGRPEWWMDYQ

SEQ ID NO: 22 - protein, 22 aa, Homo sapiens
FAEALPSDEEGESYSKEVPEME

SEQ ID NO: 23 - protein, 29 aa, Homo sapiens
FAEALPSDEEGESYSKEVPEMEKRYGGFM

SEQ ID NO: 24 - protein, 7 aa, Homo sapiens
YGGFMRF